(19) United States
(12) Patent Application Publication
(10) Pub. No.: US 2016/0331563 A1
(43) Pub. Date: Nov. 17, 2016

(54) PROSTHETIC DEVICE WITH SOCKET PRESSURE MONITORING CAPABILITY AND SYSTEMS AND METHODS FOR MAKING CUSTOM PROSTHETICS

(71) Applicant: FOOT INNOVATIONS, LLC, Coatesville, PA (US)

(72) Inventors: Justin M. KANE, Coatesville, PA (US); Mariah Anne CODY, Oyster Bay, NY (US); Kayla Marie DELANEY, Ithaca, NY (US); Laura Isabel Delgado GARCIA, Caldwell, ID (US); Istvan MORITZ, Troy, NY (US); Darren Kailung JENG, Marlboro, NJ (US)

(21) Appl. No.: 15/155,608

(22) Filed: May 16, 2016

Related U.S. Application Data
(60) Provisional application No. 62/161,678, filed on May 14, 2015.

(51) Int. Cl. A61F 2/80; A61F 2/60

(52) U.S. Cl. CPC A61F 2/80; A61F 2/60; A61F 2002/607; A61F 2250/008; A61F 2250/0001; A61F 2250/0002

(57) ABSTRACT

A prosthetic device adapted to be worn on a lower limb of a patient is provided. The prosthetic device includes a socket having an inner surface of which contacts the limb of the patient when the prosthetic device is worn by the patient. Pressure sensors are provided on the inner surface of the socket which measure pressure at the socket-limb interface when the prosthetic device is worn by the patient. The prosthetic device also includes a processor and a wireless transceiver. The processor receives data from the pressure sensors and wirelessly transmits the data to a remote wireless device. A system including the prosthetic device and a remote wireless device is also provided. The remote wireless device can display a map of pressure as a function of sensor location at the socket-limb interface and issue a warning to the patient if the pressure at a sensor location exceeds a specified value. Methods of using the prosthetic to monitor pressure at the socket-limb interface are also provided.

### CROSS REFERENCE TO RELATED APPLICATIONS

This application claims the benefit of Provisional U.S. Patent Application Ser. No. 62/161,678, filed May 14, 2015, pending, which is incorporated by reference herein in its entirety.

### BACKGROUND

1. Field

This application relates generally to a prosthetic device having socket pressure monitoring capability and systems and methods for making custom prosthetics by monitoring socket pressure during use.

2. Background of the Technology

Lower limb amputees face many challenges when obtaining a proper fitting prosthetic. Each limb has a unique topography which must be closely approximated in order to avoid further clinical issues and discomfort. Science has progressed to obtain a sufficient fit for a limb initially. However, the volume and topography of the limb can change as new scar tissue is formed. Fit can even change throughout the day after healing is complete. Factors such as activity level and weather can force fluid out of the limb, decreasing the volume. As the volume changes, the fit of the prosthetic no longer approximates the limb which can lead to compression of the tissue. This in turn can lead to irritation, inflammation, fluid buildup, and even ulcers. Lower limb amputees are more at risk of skin deterioration compared to upper limb amputations due to the high amounts of forces experienced by the limbs while walking, running, and standing.

The prosthetic socket is the interface between the amputee's residual limb and prosthetic. Unlike the tissues under the feet, the tissues of the residual limb are not able to tolerate weight-bearing loads and this often results in discomfort and ulcerations for the patient. As every amputee has a different residual limb, it makes it difficult to produce a comfortable socket that can appropriately distribute pressure across the interface. The design of the socket must account for and limit pressure at load-intolerant regions, such as areas high in bone density, while avoiding the creation of high pressure points [1]. In previous research studies, pressure had been measured in young male adults and children to better understand pressures at the socket-limb interface [2]. In general, under minor activity, patients experienced internal t to address, when taking into account the fact that the volume and the shape of the residual limb change overtime. Throughout the course of the day, the volume of the residual limb can shrink 10%-14% [3]. Other longer-term changes also occur due to weight-gain or loss and muscular atrophy.

The biomechanics of the socket-residual limb interface, especially the pressure and shear stresses, affects the fit and comfort of the prosthesis. Measuring these forces accurately is important as they can damage the limb tissue and cause ulcers. Although there are devices for measuring pressure in the socket, few are capable of accurately measuring pressure in areas of high curvature or able to measure shear stress. Providing a system that could measure these forces would aid in designing and fitting a more comfortable prosthesis.

The market for prosthetics is continuously growing with the rise of younger amputees looking into newer prosthetic designs to suit their more active lifestyle. These more advanced prosthesis are much more expensive from conventional ones. There is also a rise of diabetics which contribute to the increase in amputee population and in turn possible prosthetic users.

Accordingly, there exists a need for improved prosthetic devices, particularly prosthetic devices that are capable of monitoring the pressure at the limb-socket interface and reporting the pressure data to the patient and physician.

### SUMMARY

A prosthetic device adapted to be worn on a lower limb of a patient is provided which comprises:

a socket having an inner surface and an outer surface, wherein the inner surface contacts the limb of the patient to form a socket-limb interface when the prosthetic device is worn by the patient;

a plurality of pressure sensors on the inner surface of the socket adapted to measure pressure at the socket-limb interface when the prosthetic device is worn by the patient;

a processor; and

a wireless transceiver;

wherein the processor is adapted to receive data from the pressure sensors and wirelessly transmit the data to a remote wireless device via the wireless transceiver.

A method of monitoring pressure between a patient's lower limb and a socket of a lower limb prosthetic is also provided which comprises:

providing a prosthetic device as set forth above;

providing a remote wireless device adapted to communicate wirelessly with the wireless transceiver of the prosthetic, wherein the remote wireless device is running a software application adapted to display information regarding the data from the pressure sensors on a display of the remote wireless device;

transmitting data from the pressure sensors wirelessly to the remote wireless device as the prosthetic is being worn by the patient; and

analyzing the data.

A system for monitoring pressure between a patient's lower limb and a socket of a lower limb prosthetic is also provided which comprises:

a remote wireless device running a software application; and

a prosthetic device, the prosthetic device comprising:
a socket having a limb contacting surface;
a plurality of pressure sensors on the limb contacting surface;
a processor; and
a wireless transceiver;

wherein the pressure sensors are adapted to measure pressure at the socket-limb interface;

wherein the processor is adapted to receive data from the pressure sensors and wirelessly transmit the data to a remote device via the wireless transceiver; and

wherein the software application is adapted to display information regarding the data on a display of the wireless device.

These and other features of the present teachings are set forth herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a schematic showing a vertical cross-section of a lower limb prosthetic device showing the socket (top of drawing) and a plurality of pressure sensors disposed an an inner surface of the socket.

FIG. 2 is a schematic showing a perspective view of a lower limb prosthetic device showing a plurality of antennas disposed on an outer surface of the socket and a housing for the electronics secured to the prosthetic.

FIG. 3A is a schematic showing a left-facing view of a residual lower limb showing various locations for pressure sensor placement.

FIG. 3B is a schematic showing a front-facing view of a residual lower limb showing various locations for pressure sensor placement.

FIG. 3C is a schematic showing a right-facing view of a residual lower limb showing various locations for pressure sensor placement.

FIG. 4 is a schematic illustrating a prosthetic device as described herein being worn by a patient wherein the prosthetic device transmits pressure readings from the socket-limb interface to a remote wireless device such as a smart phone or a computer.

FIG. 5 is a schematic illustrating a housing for the electronics of the device secured to a prosthetic, wherein the device is shown with a power switch, a power indicator and a battery compartment cover.

### DETAILED DESCRIPTION

As used herein, a “remote wireless device” is a device located remotely from the prosthetic that is capable of communicating wirelessly with the wireless transceiver on the prosthetic device. The remote wireless device can be a smart device including, but not limited to, a smart phone or a tablet. The remote wireless device can also be a laptop or desktop computer with wireless capability.

As used herein a “specified value” is a value of a variable (e.g., pressure) provided by a clinician or otherwise input into the prosthetic device or software application running on the remote wireless device. For example, the pressure measured at a sensor location can be compared to a specified value of pressure which corresponds to a pressure level which should not be exceeded to ensure safe use of the prosthetic device. The specified value can be a cumulative value (e.g., an aggregate value of a variable taken over a period of time) such as cumulative pressure.

As used herein a “specified period of time” is a period of time provided by a clinician or otherwise input into the prosthetic device or software application running on the remote wireless device. Pressure readings can be taken over a specified period of time and compared to a specified cumulative value of pressure which corresponds to a cumulative pressure level which should not be exceeded to ensure safe use of the prosthetic device.

As used herein, “MEMS” refers to Micro-Electro-Mechanical Systems which are devices that include miniaturized mechanical and/or electro-mechanical elements that can be made using the microfabrication techniques. MEMS can be made up of components between 1 to 100 micrometers in size (i.e., 0.001 to 0.1 mm), and MEMS devices can range in size from 20 micrometers to a millimeter (i.e., 0.02 to 1.0 mm).

As used herein, “BioMEMS” refers to biomedical micro-electro-mechanical systems (MEMS). BioMEMS include MEMS that are used in or suitable for use in biomedical applications.

According to some embodiments, a prosthetic device having socket pressure monitoring capability and systems and methods for making custom prosthetics by monitoring socket pressure during use are provided.

According to some embodiments, prosthetic devices retrofitted with sensors, including pressure sensors which can communicate wirelessly with a computer or smart device are provided. The data can be accessed by a physician who can communicate information to the patient. Power to the device can be supplied via a rechargeable battery or using kinetic energy (i.e., movement of the patient wearing the prosthetic).

According to some embodiments, systems and methods are provided which comprise one or more of the following features/characteristics:

1) Monitor pressure at the socket/limb interface and model it in a 3-dimensional format.

2) Allow for an interface with the practitioner to model a prosthetic having a socket that appropriately fits and addresses the underlying medical condition.

3) Allow for the modelling software to use automation to model the prosthetic based off of the data accrued from #1.

4) Create an output file to render a 3d printed medical device from the data accrued in #1 and the modelling input from #2 and #3.

5) Allow for real time monitoring of pressure by providing the prosthetic with pressure sensors that can measure pressure over the course of the gait cycle.

6) Qualitatively measure pressure over the course of time as the prosthetic is being worn by the patient.

7) Quantitatively measure pressure over the course of time as the prosthetic is being worn by the patient.

8) Store the data with respect to #6 and #7 in a cloud based format.

9) Notify the person wearing the device of the data in #6 and #7 as predefined by a practitioner.

10) Notify the practitioner of all data in #6 and #7.

11) Data can be transmitted wirelessly. Notification can be, for example, via Bluetooth, WiFi, and/or via personal smartphone application.

12) Track pressure in real time in order to monitor cumulative pressure over the course of time.

13) Transmit feedback to patient from #12 in order to assure patient is not experiencing momentary or cumulative pressure overload.

14) Transmit feedback to practitioner from #12 in order to assure patient is not experiencing momentary or cumulative pressure overload.

According to some embodiments, a pressure mapping system is implemented in order to obtain a three dimensional image of the pressures experienced on the prosthetic-limb interface. This pressure data allows the patient to adjust the prosthetic accordingly to decrease the pressure in key areas.

A prosthetic device is provided which comprises a socket that dynamically measures pressure at the socket-limb interface to allow patients to actively improve their own health care and prevent ulcerations. This device can help improve the comfort and safety of prosthetics for all lower limb amputees, especially those with higher levels of activity, and more specifically, patients such as veterans and children who suffer from birth defects and other diseases which lead to amputations. These patients are younger and in most cases want to engage in a more active in order to have a happy, fulfilling life.

Patients using the prosthetic device will be able to actively monitor the fit of their prosthetic. This will grant patients an increase in mobility while also potentially decreasing the amount of doctor visits the patient must undergo. In the long term, this will decrease healthcare costs and allow patients to be more actively involved in their own care.

As set forth above, lower limb amputees are prone to ulcerations and general discomfort. The volume and topography of the limb change as new scar tissue is formed, and current prosthetic technologies cannot change dynamically along with the residual limb. To address this clinical need, pressure can be measured at the socket-limb interface, and the measured pressure can be used to inform the patient, over time, of elevated pressures that could lead to ulcerations.

The prosthetic with self-monitoring capabilities can also be used as a research tool for physicians to quantify experienced pressures. The prosthetic can also be used to develop a prosthetic socket that changes dynamically with the patient's residual limb in response to the pressure data, thereby preventing ulcerations and discomfort. This will allow patients an increase in mobility and decrease the amount of doctor visits the patient must undergo. In the long term, this will decrease healthcare costs and allow patients to be more actively involved in their own care.

According to some embodiments, the device measures pressure over at least 90% of the area of the entire socket-limb interface. According to some embodiments, the device measures pressure over at least 95% of the area of the entire socket-limb interface. According to some embodiments, the device can alert the user to adjust the fit of the socket if abnormal pressure is detected.

The sensors should be fixed to the socket inner surface without hindering the normal phases of gait and adapted to withstand normal wear. According to some embodiments, the device can record data continuously for a full day after charge. Data can be communicated wirelessly or via a wired connection (e.g., USB) to a computer for further examination. The device should be able to withstand any weather conditions the user may encounter during use.

According to some embodiments, the device complies with Part 15 of the FCC Rules deadline with low power non-licensed transmitters. According to some embodiments, operation of the device does not cause harmful interference, and the device accepts any interference received, including interference that may cause undesired operation.

According to some embodiments, the device is able to measure pressure with an accuracy of at least 95% at all locations via sensors. According to some embodiments, the device has a resolution of at least 4 sensors per in². According to some embodiments, the device has a resolution of at least 10 sensors per in².

Along with achieving this level of accuracy and spatial resolution, the device should also be reliable and durable. This requires the device to be able to withstand perspiration from the user and all weather conditions, including precipitation, while maintaining full functionality. The sensors should be affixed or secured so as not to be dislocated during use, as movement of the sensors would affect the readings.

According to some embodiments, the device can function for a full day on a single charge. Since the average lower limb amputee sees their physician semi-annually, the device should not require maintenance for at least 6 months.

The lithium ion battery should be replaced every three years for optimal performance. Lithium ion batteries are expected to last between two to three years before performance starts to diminish. The temperature range for the battery is between −20° C. and 60° C. [5]. The battery compartment of the device should be accessible to allow for simple battery replacement. As the patient visits their clinician semi-annually, the clinician can verify the validity of the measured pressures and recalibrate the device if necessary.

The wireless pressure networks used to monitor pressure can be packaged individually in generic plastic packaging. Since the final product will not contact patient's skin, no sterilization practices would generally be required.

The inside of the socket should be washed with antibacterial soap and hot water daily. An antiperspirant spray or deodorant can be applied on the stump if required. To protect the sensors from sweat, a liner will be placed over the sensors in the socket. The liner can be removable to allow it to be easily cleaned.

The device should be able to withstand all weather conditions. To accommodate this, this device will be designed to be durable and water resistant to allow patients to utilize the final design in a variety of weather conditions. The electronic components can be located around the calf portion of the lower limb, encased in a protective plastic shell casing. The antenna receivers can be coated in a waterproof primer.

The device is intended to be used on prosthetics designated for walking. Accordingly, the device should not affect overall mechanical properties of the prosthetic. This means that the device will withstand daily loading.

The primary interaction of the device with the user occurs at the socket-limb interface. In this interaction, the sensors are not in direct contact with the patient's residual limb. To ensure the greatest comfort, the sensors should be thin. According to some embodiments, the sensors have a thickness that does not exceed 0.007 inches. To further improve comfort, the sensors should be fixed in a manner that allows for a smooth finish. According to some embodiments, a surface primer that is compatible with the plastic used for the socket cup can be used to affix the sensors.

The user may also need to interact daily with the lithium ion battery. The battery used to power the device can be a lithium ion battery similar to the batteries used to for cell phones and other USB compatible devices.

The design of the socket interface will vary in size as the morphology of each patient's residual limb is different. There are restrictions on the width and placement of the sensors. According to some embodiments, the final width on the inside of the prosthesis should not exceed 0.008 inches and should be completely smooth at the surface. The center pin hole of the prosthetic should remain uncovered to allow the patient to seamlessly fit their prosthetic.

All of the components should be easily accessible to allow for maintenance. The battery is directly accessible to the patient to allow them to charge the final design daily (e.g., overnight) [5].

The weight of a typical below the knee prosthetic is 3.75 lbs, which is about half the weight of a natural limb at 7 lbs for a 115 lb person [4]. A goal of keeping the pressure mapping system under 3 lbs would keep the device below the weight of a typical limb and should not impair the usage of the prosthetic. However, the ultimate goal is to keep the weight of the system as low as possible. Minimizing weight will make the handling of the prosthetic easier for the user and will reduce the effect that added weight may have on the performance of the prosthetic.

There are very few restrictions on the materials that can be used for manufacturing the device. Biocompatibility is not a concern when selecting materials since the device is an external device and none of the materials used will be in direct contact with any body tissue. The materials used for the electronics will be housed and secured to the prosthetic (e.g., in the calf area) and the sensors will be covered on the inside of the prosthetic socket thereby providing some protection to the electronic components. The materials used for the casing and the socket liner should, however, be corrosion resistant as the device will be used outside and should not be damaged by various weather conditions. The liner material used to cover the sensors also must be able to bind composites and resins with polyethylene in order to ensure that the sensors are properly secured to the inside of the socket. That being said there are few restrictions on materials, with proper sealing of the casing being more of a concern for weather resistance.

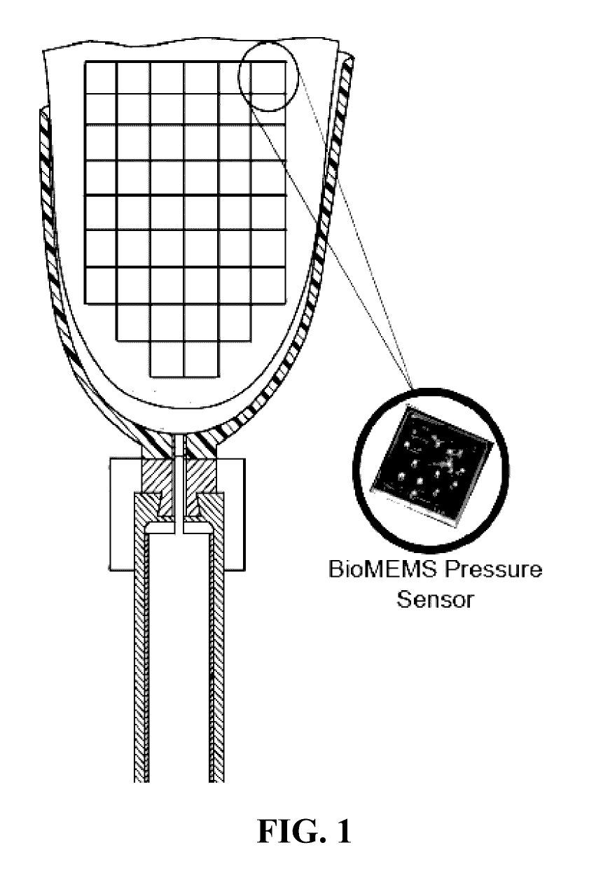

FIG. 1 is a schematic showing a vertical cross-section of a portion of a lower limb prosthetic device showing the socket (top of drawing) and a plurality of pressure sensors disposed on an inner surface of the socket. As depicted in FIG. 1, the pressure sensors are wireless BioMEMS pressure sensors which do not require a wired connection (e.g., to a power source or antenna). The pressure sensors are disposed in an array to monitor pressure over the surface of the limb-socket interface.

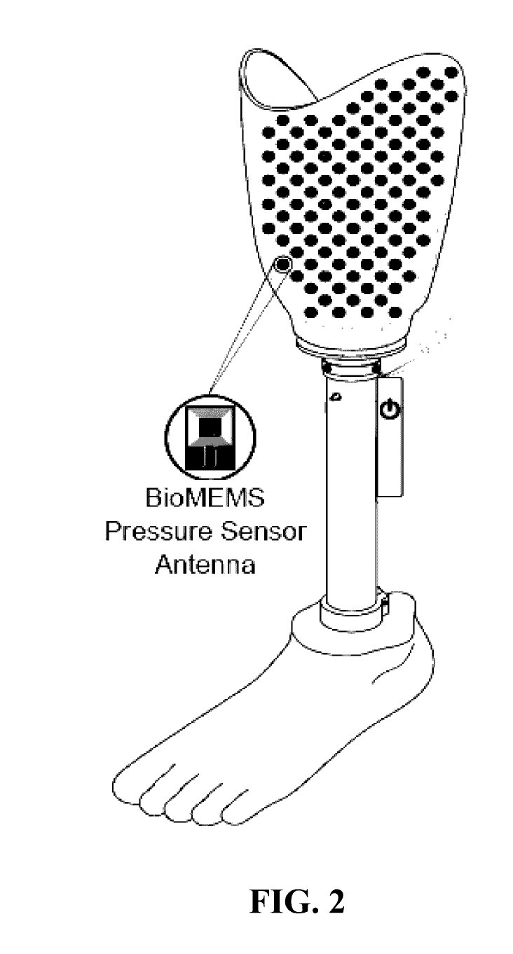

FIG. 2 is a schematic showing a perspective view of a lower limb prosthetic device showing a plurality of antennas disposed on an outer surface of the socket and a case for the electronics of the device secured to the prosthetic. A power button is shown on the side of the case. As depicted in FIG. 2, the antennas are wireless BioMEMS pressure sensor antennas disposed in an array. As also depicted in FIG. 2, the antennas are connected via wires to the electronics in the case. The pressure sensors on the inner surface of the socket (not shown in FIG. 2) communicate wirelessly with a corresponding antenna on the outer surface of the socket. Pressure data from each sensor is then transmitted via a wired connection to a processor in the case.

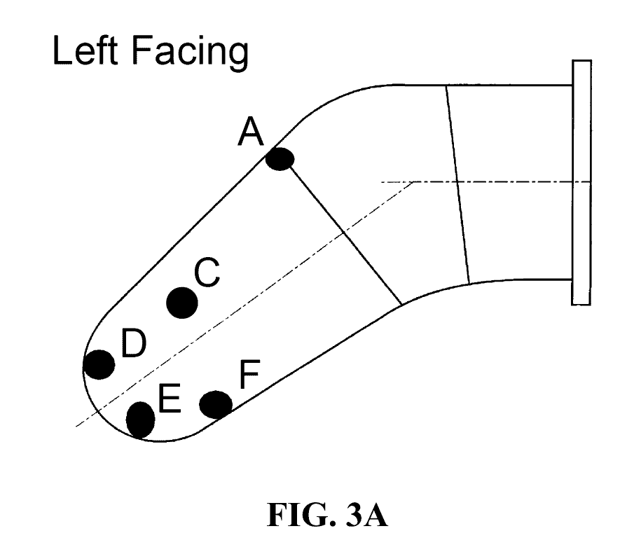

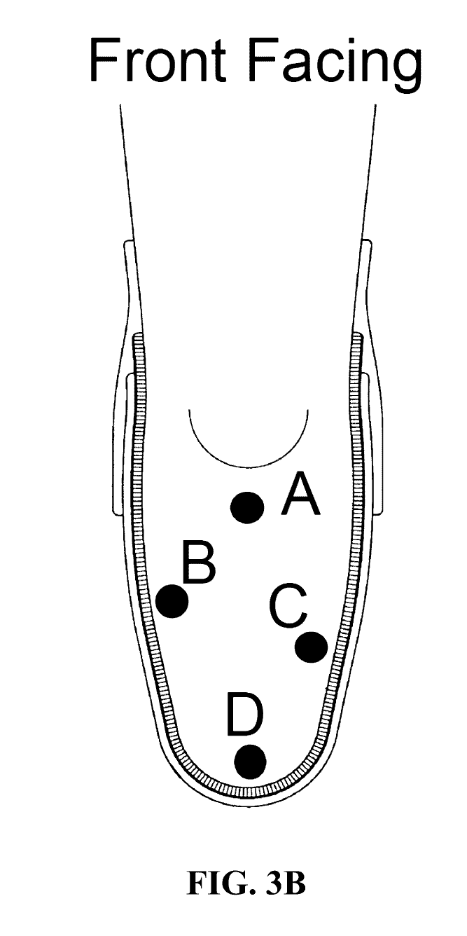

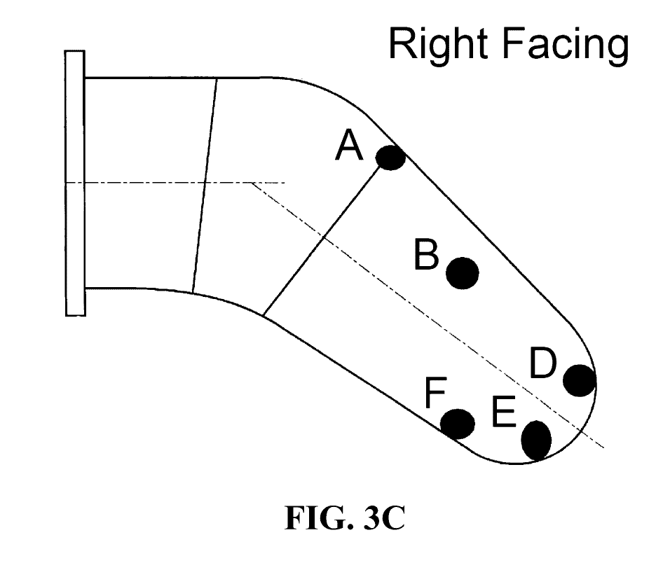

FIG. 3A is a schematic showing a left-facing view of a residual lower limb showing various locations for pressure sensor placement. FIG. 3B is a schematic showing a front-facing view of a residual lower limb showing various locations for pressure sensor placement. FIG. 3C is a schematic showing a right-facing view of a residual lower limb showing various locations for pressure sensor placement. According to some embodiments, sensors can be placed at one or more of the indicated locations.

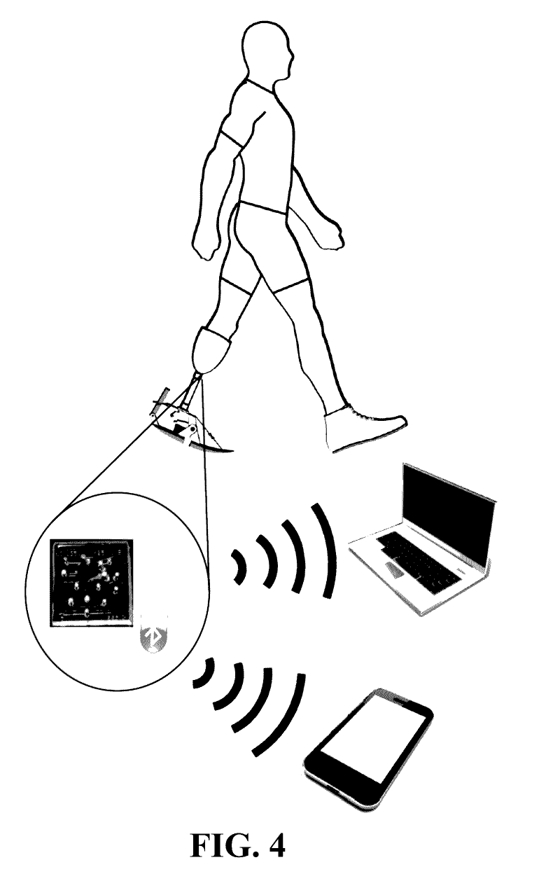

FIG. 4 is a schematic illustrating a prosthetic device as described herein being worn by a patient wherein the prosthetic device transmits pressure readings from the socket-limb interface to a remote wireless device such as a smart phone or a computer. As shown in FIG. 4, the remote wireless device can be a laptop computer or a smart phone.

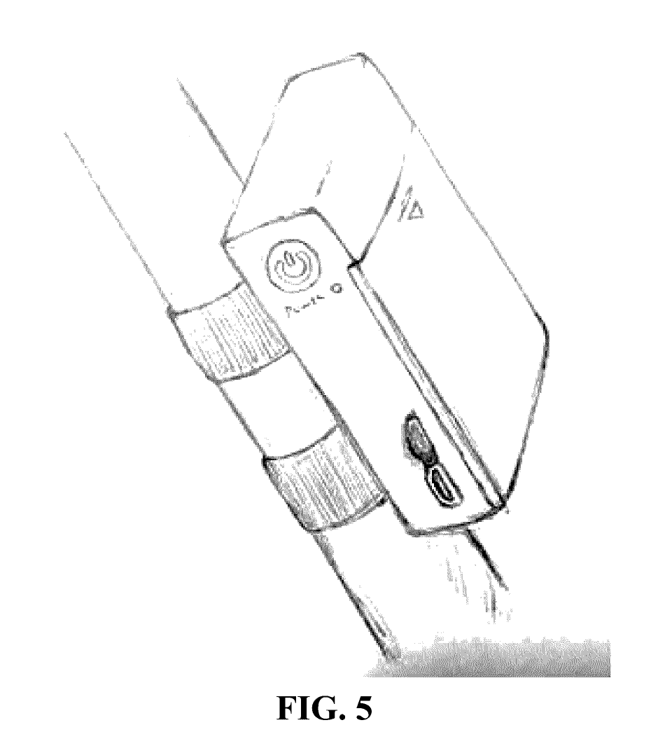

FIG. 5 is a schematic illustrating a case for the electronics of the device secured to a prosthetic, wherein the device is shown with a power switch, a power indicator (e.g., an LED light) and a battery compartment cover. A charging port for the battery is also depicted at the bottom on the side of the case below the power switch. A wired connection (e.g., a USB port) can also be provided for downloading the data to an external device.

The sensors can be laminated onto the socket interface with minimal thickness. The surface should be smooth to provide minimal resistance and irritation while fitting and wearing the prosthesis. The outside antennas can be coated with a waterproof primer. The coating can match the socket's color to provide a cohesive look. As shown in FIG. 5, the electronics can be encased and attached securely to the rod of the prosthetic. The power button and charging port are located on the side of the device; the LED indicates power/charging status. The charging port has a cover for waterproofing. The front cover slides out for battery access. The small profile of the device, paired with a matte finish, makes the device sleek and sexy.

The final design can be retrofitted into existing prosthetic solutions. The circuitry unit along with the casing, the Bluetooth transceiver and microcontroller can be printed and produced in bulk. The pressure sensors, antennas, laminate, and batteries can also be produced or acquired in bulk. The pressure sensors can be arranged in wireless network grids.

The housing that protects the electronics can be a made using a variety of manufacturing techniques including, but not limited to, injection molding, 3D printing and thermoforming. Polypropylene can be used as a casing material for the material due to its low density, good mechanical properties and its flexibility for hinged parts. The housing can be a relatively small plastic box (e.g., 4″×1″×1″) and can include a band to allow it to attach to the metal support shaft that connects the socket to the foot.

Table 1 is a table which provides some exemplary device specifications. These device specifications are exemplary only and are not intended to be limiting. Many of those specifications relate to the comfort of the patient, the amount of pressure readings taken, how readings are taken accurately, and the different aspects of daily routines the device must be able to withstand. In addition, there are other device specifications: 1) the device can be small so that it can be easily concealed; 2) the device can be easily accessible to the user for charging; 3) the device can notify the patient when high levels of pressure are reached in a specific area; 4) the socket-limb interface of the device can be easily cleaned; 5) the device can operate in all weather conditions. These additional specifications can be addressed as follows: 1) by concealing the device inside the calf, thus limiting the size of the device; 2) the charger can be made accessible through an opening in the device casing; 3) the device can wirelessly send a message via text or email to the end user; and 4) a liner material can be used to protect the electronics from moisture and allow for quick, easy cleaning.

TABLE 1
A Summary of Exemplary Device Specifications

| | | Exemplary |
|---|---|---|
| | Metrics | Specification |
| Device should give an accurate reading of | Spatial Resolution | at least 4 Sensor per in² |
| entire socket-limb interface | (sensors/in²) | |
| Device should give an accurate reading for | Sensing Area (in²) | 3 in × 8 in |
| entire socket-limb interface | | |
| Device should not be noticeable or irritate | Thickness (in) | <.007 in |
| skin of the limb | | |
| Device should continuously measure | Sampling Rate (Hz) | >=10 Hz |
| pressure throughout daily movements | | |
| Device should be able to measure pressure | Pressure Range (kPa) | 40-70 kPa |
| ranges experienced in daily movements | | |
| Device should be able to give an accurate | Accuracy (%) | +/−5% |
| reading of pressure experienced | | |
| Device should be able to function in all | Temperature Range (° F.) | 0-100° F. |
| weather conditions | | |
| Device should not create a noticeable | Weight (lbs) | <3.5 kg |
| difference while performing daily | | |
| movements compared to prosthetics | | |
| without the device | | |
| Device should not be significantly more | Cost ($) | <$17,000 |
| than the cost of several iterations of | | |
| prosthetics | | |
| Device should continue to function as long | Shelf Life (years) | >3 years |
| as the prosthetic is used | | |

Spatial resolution refers to how many sensors are placed per unit area. Spatial Resolution can be gathered from benchmarking existing systems. Sensing area refers to the area of the socket-limb interface in which pressure readings are taking place. Sensing area can be calculated from measuring the width and height of an average prosthetic socket. Thickness refers to the thickness of the pressure sensor. Thickness can be identified from the thickness causing discomfort through the average sock that covers the limb when fitting a prosthesis. Sampling rate refers to how often pressure readings are taken. Pressure range refers to what pressures the sensors can measure. Pressure range can be calculated using pressure data from previous research studies. Sampling rate can be determined by testing how long the average person goes through the three phase gates of both walking and running. If the information gathered falls within a 95% confidence interval, the device may be more useful for research and information. Accuracy refers to the percent difference between the measure output of pressure and the actual pressure experienced on the limb. Temperature range refers to the temperatures at which the device will function. The temperature range can be obtained from average weather conditions across the United States. The weight of the device refers to the weight of all the components of the device. The maximum weight can be measured from weight of the prosthetic with a tolerance of a ½ kg. Below this tolerance, the patient's use of the prosthetic should be unaffected. Cost refers to the entire manufacturing and medical costs of implementing this device. The Shelf life refers to how long the device can remain in working condition before one or more of the components need replacing.

Every patient has a unique residual limb which results in a variation of preferences and essentials when selecting a type of prosthetic. Factors which affect what type of prosthetic to be selected include location of amputation (above or below the knee), the activity level of the patient, the cause of amputation (injury or disease), and the amount of sensation of the residual limb. In general, patients want a prosthetic which is easy to maneuver, easy to adjust, and cause the least amount of pain. Patients do not want prosthetics which inhibit movement, require many adjustments throughout the day, and are expensive.

Patients may choose between different modes of attachment. Two modes of attachment that can be used for the prosthetic include a pin based system and a suction based system. After speaking with different patients, it was clear that each had their own opinion on which method was best. More active patients tend to prefer the suction based system due to its superior secure attachment to the residual limb. Less active patients tend to prefer the pin based system due to its ease of attachment and removal and since it is less restricting compared to the suction-based system. If the amputation is just below the knee, the patient can sometimes experience limitations on knee mobility due to the high compression of the suction-based system. This can result in discomfort and for more active patients this can result in limitations of activities.

Patients with above the knee amputations are especially concerned with maneuverability due to the laborious demands of using common prosthetics. In addition to moving the limb forward, an extra motion is require in order to straighten the knee. This can require a lot of practice to implement properly and does not allow for quick movements. Patients also find wearing prosthetics at all to be uncomfortable due to the high compression of the socks which are worn. For above the knee prosthetics, the socks may be high up the leg causing compression in the groin area. Both of these concerns can lead above the knee amputees to not wearing prosthetics and relying on wheelchairs for mobility.

According to some embodiments, a device is provided which can be used for below the knee amputees. According to some embodiments, a device is provided which can be used by moderate to high activity level amputees.

According to some embodiments, patients are able to receive readings at the socket-limb interface while wearing the prosthesis throughout the day. According to some embodiments, the system communicates wirelessly (e.g., via Bluetooth) to the patient's phone, computer or other smart device. According to some embodiments, when the socket-limb interface is experiencing dangerous pressure readings, the patient will be alerted via text messaging or email service. According to some embodiments, the patient will have access to a pressure map ranging from green/blue to red, where red indicates dangerous pressure readings. Providing a pressure map will allow the patient to discern where abnormal pressures are located and prevent possible ulcerations from occurring.

Consumers, if given the knowledge that a pressure sensing system will aid in the design of new dynamic sockets, will adopt the technology. Many prosthetic users, especially those older and under Medicare, are in need of a “one prosthesis for life.” These permanent prosthesis will most likely use a dynamic socket to circumvent the prevalent issue of prosthetic fitting. With the rising interest of dynamic sockets, this device can be marketed as a research tool for prosthesis companies to use in their development departments.

According to some embodiments, a prosthetic is provided which comprises a socket having a limb contacting surface and a sensor network of MEMS-based sensors configured into a pressure map on the limb contacting surface. The measured pressures can be transmitted wirelessly (e.g., via Bluetooth) to the patient's smartphone or other smart device. An application (i.e., app) on the smart device can be used to display a 3-dimensional representation of the socket-limb interface, based on the measured pressure. The app can also show the patient where any damage-causing pressures are being experienced, and alert the patient via a text message or email, when these dangerous pressures occur, so that the patient may manually adjust the fit of their socket to relieve the pressure, either by simply removing the prosthetic for a short period of time or removing/adding socks to account for any volume change of the residual limb. The sensors and Bluetooth transceiver can be powered by a battery (e.g., a lithium ion battery) and housed in a waterproof casing attached to the prosthetic.

Each sensor can be paired with an antenna to allow for wireless communication and powering of the sensors via a Bluetooth transceiver chip. The antennas can be attached to the outer wall of the socket, and a layer of primer can be evenly coated over the antennas to protect them from the elements.

According to some embodiments, a circuit containing a microcontroller chip with an analog to digital converter, a Bluetooth transceiver chip, and a multiplexer for the sensors can be printed. This printed circuit board can be housed in a rectangular unit (e.g., 4″×1″×1″) to protect and secure the electronics. The housing can be made from a thermoplastic material via any suitable manufacturing technique, including, but not limited to, injection molding, 3D printing and thermoforming. Suitable materials for the housing include, but are not limited to, polypropylene. Polypropylene has a relatively low density, good mechanical properties and good flexibility for hinged parts. The housing unit can be attached to the prosthetic. For example, the housing can be attached to the metal support shaft that connects the socket to the foot via a belt system.

If the patient is making an entirely new prosthesis, the surface of the residual limb can be scanned electronically to account for placement of sensors on the surface of the prosthesis to allow for a completely flat interface. If the patient is retrofitting a pressure monitoring system into their current prosthesis, the orthopedic surgeon can hollow out a uniform layer at the prosthetic surface to allow for a completely flat interface after installation of the sensor network.

The liner material can be a combination of Duratec Gray Surface Primer and FibreGlast Polyester Gel Coat. The Duratec Gray Surface Primer adheres to fiberglass, resins, and polyethylene, allowing the sensors to be fixed onto the prosthetic interface while providing a smooth, polished surface without compromising thickness. The FibreGlast Polyester Gel Coat is optional. The gel coat allows for the creation of a glossy finish in any color, while not affecting the fit or performance of the device. Both the primer and gel coat (if used) can be applied using a standard pressurized spray can with the advantage of having fast room temperature curing, allowing for a quick and simple application.

In order to collect analog data from the sensors an analog to digital converter (ADC) can be used. The Texas Instruments MSP430G24521N20, is a standalone microcontroller, which contains a 10 bit analog to digital converter (ADC) with 8 channels. This controller can be a part of a dedicated circuit, which can connect the power source (battery), the sensors, and a Bluetooth transceiver, such that data from the sensors can be read and transmitted wirelessly to a computer or smartphone. To communicate with more than eight sensors, a multiplexor can be used. A switch can also be incorporated into the circuit as well as a charging port, to allow for users to turn on and off the device as well as charge it.

Bluetooth communication is common in most smartphone and computers. To communicate the device to a smartphone/computer with relative ease, a Bluetooth transceiver can provide this capability. The Bluetooth connectivity does not require the use of external wires, a wireless network, nor a cell phone data plan; it is stand-alone communication. The use of Bluetooth does not burden the device as it draws a low amount of current.

An electronic casing can provide housing and protection for the microcontroller, battery, and other circuitry. The casing can be a watertight plastic enclosure. The casing can be tested to Ingress Protection (IP) standards of IPx7. The housing can be water resistant to depths of up to 1 meter. The casing can be tested by submerging the housing in one meter of water for 30 minutes and inspecting for leaks. The casing can also contain access ports for charging and turning the pressure monitoring system on and off.

The chosen design offers a couple of key advantages: the design can be retrofitted into any prosthetic; the components for the design are lightweight and inexpensive; the design is comfortable for the patient.

The most significant advantage of the chosen design is that it can be retrofitted into any prosthetic. This allows a patient or physician to purchase this device and implant it without the cost of an entirely new lower limb prosthetic unit. This means that the final design will be available to patients that already own their prosthetic limb. This is extremely important, because the final design will be used as a research tool for orthopedic physicians to understand pressures that are occurring at their patient's socket-limb interface to avoid ulcerations.

The overall weight and size of the final design prototype should not affect the use and wearability of the patient's prosthetic. The sensors and any coating bused to cover the sensors (e.g., primer) should be thin so that the sensors placed in the interior of the socket cannot be detected and do not cause any irritation or skin damage to the patient.

If wireless sensors are used, there will be no need for any wiring on the inside of the socket, which will prevent the need of dongles such as seen in the TekScan F-system and further reduce the likelihood of irritation. Use of wireless sensors will also help maintain the integrity of any suction mechanism used to attach the sock to the socket. The small size electrical components can be discretely placed inside the calf of the prosthetic without any change to the external shape. The electrical components are lightweight so there is no noticeable difference in movement of the prosthetic limb pre and post device implantation.

MEMS based wireless pressure sensors can be used. Using wireless sensors eliminates the need for wires in the socket thereby allowing for a more comfortable pressure monitoring system than wired sensors.

The sensors may need to be calibrated when implanted in a socket. All residual limbs have a unique topography which may cause each sensor to have different curvature against the socket surface. The sensors can also be covered by a laminating material to affix them to the socket and protect them from damage. Both the unique topography and the laminating of the sensors can affect the readings thereby requiring calibration.

While the foregoing specification teaches the principles of the present invention, with examples provided for the purpose of illustration, it will be appreciated by one skilled in the art from reading this disclosure that various changes in form and detail can be made without departing from the true scope of the invention.

### REFERENCES

[1] Engsberg, J. R. (2015, Feb. 21). Quantifying Interface Pressures in Below-Knee-Amputee Sockets. Retrieved from ACPOC: http://www.acpoc.org/library/992_03_081.asp.
[2] Sanders, J. E., & Daly, C. H. (1993). Normal and shear stresses on a residual limb in a prosthetic. Journal of Rehabilitation Research, 191-204.
[3] “Get in Contact.” Keeping Your Leg on. N.p., n.d. Web. 21 Feb. 2015. http://www.ottobockus.com/prostlietics/info-for-new-amputees/prosthetics-101/keeping-your-leg-on-(suspension)/
[4] “How Much Does Your Leg Weigh?—Stanmore User Group.” How Much Does Your Leg Weigh?—Stanmore User Group. Stanmore Limb User Group, n.d. Web. 19 Apr. 2015. http://www.stanmorelimbusers.co.uk/information-advice/how-much-does-your-leg-weight
[5] “Battery Storage.” Electropaedia. N.p., n.d. Web. 1 May 2015. http://www.mpoweruk.com/storage.htm>.

What is claimed is:

1. A prosthetic device adapted to be worn on a lower limb of a patient, the prosthetic device comprising:
a socket having an inner surface and an outer surface, wherein the inner surface contacts the limb of the patient to form a socket-limb interface when the prosthetic device is worn by the patient;
a plurality of pressure sensors on the inner surface of the socket adapted to measure pressure at the socket-limb interface when the prosthetic device is worn by the patient;
a processor; and
a wireless transceiver;
wherein the processor is adapted to receive data from the pressure sensors and wirelessly transmit the data to a remote wireless device via the wireless transceiver.

2. The prosthetic device of claim 1, wherein the pressure sensors are wireless, wherein each pressure sensor is paired with an antenna and wherein each pressure sensor communicates wirelessly with a corresponding antenna.

3. The prosthetic device of claim 2, wherein each of the antennas are attached to the outer wall of the socket.

4. The prosthetic device of claim 1, further comprising a coating layer over the pressure sensors.

5. The prosthetic device of claim 1, wherein the wireless transceiver is a Bluetooth transceiver.

6. The prosthetic device of claim 1, further comprising a battery adapted to power the processor and wireless transceiver.

7. The prosthetic device of claim 6, wherein the processor, the wireless transceiver and the battery are housed in a casing and wherein the casing is secured to the prosthetic.

8. The prosthetic device of claim 7, wherein the battery is rechargeable and wherein the casing includes a charging port.

9. The prosthetic device of claim 2, wherein the antennas are connected to the processor via a wired connection.

10. A system for monitoring pressure between a patient's lower limb and a socket of a lower limb prosthetic, the system comprising:
a remote wireless device running a software application; and
a prosthetic device, the prosthetic device comprising:
a socket having a limb contacting surface;
a plurality of pressure sensors on the limb contacting surface;
a processor; and
a wireless transceiver;
wherein the pressure sensors are adapted to measure pressure at the socket-limb interface;
wherein the processor is adapted to receive data from the pressure sensors and wirelessly transmit the data to a remote device via the wireless transceiver; and
wherein the software application is adapted to display information regarding the data on a display of the wireless device.

11. The system of claim 10, wherein the software application is adapted to upload the data to a computer network.

12. The system of claim 10, wherein the software application is adapted to display cumulative pressure over a specified period of time one or more sensor locations.

13. The system of claim 10, wherein the software application is adapted to display a map of pressure as a function of sensor location at the socket-limb interface.

14. The system of claim 13, wherein the software application is adapted to display a three-dimensional representation of the map.

15. The system of claim 13, wherein the software application is adapted to display one of a plurality of different colors at each sensor location on the map depending on the pressure measured at the sensor location.

16. The system of claim 15, wherein the software application is adapted to display the color red at a sensor location on the map when the pressure is above a specified value.

17. The system of claim 10, wherein the software application is adapted to detect when a pressure exceeding a specified level at a sensor location is experienced and issue a warning to the patient.

18. The system of claim 10, wherein the warning is in the form of a text or e-mail.

19. The system of claim 10, wherein the wireless transceiver is a Bluetooth transceiver.

20. The system of claim 10, further comprising a battery adapted to power the processor and wireless transceiver.

21. The system of claim 20, wherein the processor, the wireless transceiver and the battery are housed in a casing and wherein the casing is secured to the prosthetic device.

22. The prosthetic device of claim 21, wherein the battery is rechargeable and wherein the casing includes a charging port.

23. The system of claim 10, wherein the pressure sensors are wireless, wherein each pressure sensor is paired with an antenna and wherein each pressure sensor communicates wirelessly with a corresponding antenna.

24. The prosthetic device of claim 23, wherein each of the antennas is connected to the processor via a wired connection.

25. A method of monitoring pressure between a patient's lower limb and a socket of a lower limb prosthetic, the method comprising:
providing a lower limb prosthetic as set forth in claim 1;
providing a remote wireless device adapted to communicate wirelessly with the wireless transceiver of the prosthetic, wherein the remote wireless device is running a software application adapted to display information regarding the data from the pressure sensors on a display of the remote wireless device;
transmitting data from the pressure sensors wirelessly to the remote wireless device as the prosthetic is being worn by the patient; and
analyzing the data.

26. The method of claim 25, wherein analyzing the data comprises displaying a map of pressure as a function of sensor location at the socket-limb interface.

27. The method of claim 25, wherein analyzing the data comprises comparing the pressure data at each sensor location to a specified value and issuing a warning if the pressure at a sensor location exceeds that value.
